(19) United States
(12) Patent Application Publication
(10) Pub. No.: US 2012/0190849 A1
(43) Pub. Date: Jul. 26, 2012

(54) PROCESSES FOR THE PREPARATION OF VARDENAFIL

(75) Inventors: Anu Mittal, Kurukshetra (IN); Mahavir Singh Khanna, New Delhi (IN); Rajesh Kumar Thaper, Jammu (IN); Mohan Prasad, Gurgaon (IN)

(73) Assignee: RANBAXY LABORATORIES LIMITED, New Delhi, Delhi (IN)

(21) Appl. No.: 13/389,358

(22) PCT Filed: Aug. 9, 2010
(86) PCT No.: PCT/IB10/53594

§ 371 Date: Mar. 30, 2012

(30) Foreign Application Priority Data
Aug. 7, 2009 (IN) 1650/DEL/2009

(51) Int. Cl. C07D 487/04

(52) U.S. Cl. 544/184

(57) ABSTRACT

The present invention provides processes for the preparation of vardenafil, its pharmaceutically acceptable salts, hydrates and intermediates.

### FIELD OF THE INVENTION

The present invention provides processes for the preparation of vardenafil, its pharmaceutically acceptable salts, hydrates and intermediates.

### BACKGROUND OF THE INVENTION

Vardenafil is chemically 2-ethoxy-5-[(4-ethyl-1-piperazinyl)sulphonyl]phenyl-5-methyl-7-propylimidazo[5,1-f][1,2,4]triazin-4(3H)-one and has a structure as represented by Formula I:

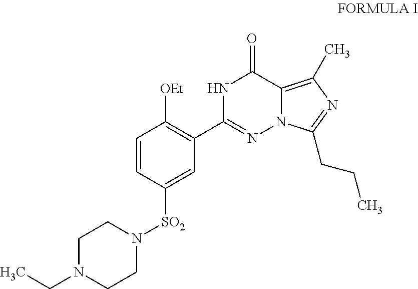

Vardenafil is known from U.S. Pat. No. 6,362,178 and is marketed as vardenafil hydrochloride trihydrate salt under the trade name Levitra®. It is a phosphodiesterase type 5 inhibitor and is indicated for the treatment of erectile dysfunction in mammals.

Several methods for the preparation of vardenafil are reported in literature such as those described in U.S. Pat. Nos. 6,362,178; 6,777,551; 7,022,847; U.S. Publication 2006/0264624; and Org. Process Res. Dev., 9(1), pages 88-97, (2005).

### SUMMARY OF THE INVENTION

In one general aspect, the present invention provides for a process for the preparation of vardenafil of Formula I,

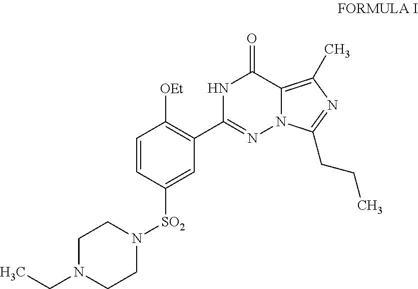

its pharmaceutically acceptable salts and hydrates. The process includes the steps of:
i) hydrogenating 2-ethoxy-N-hydroxy-5-[(4-ethylpiperazin-1-yl) sulphonyl]benzene carboximidamidine of Formula II

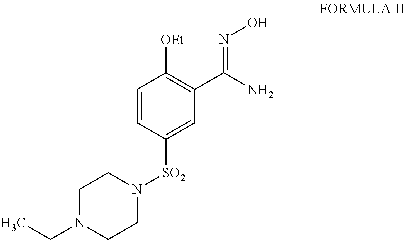

to obtain 2-ethoxy-5-(4-ethyl-1-piperazinylsulfonyl)benzamidine of Formula III

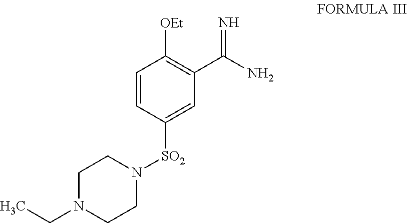

or its salt;
ii) converting the 2-ethoxy-5-(4-ethyl-1-piperazinylsulfonyl)benzamidine of Formula III or its salt to vardenafil of Formula I; and
iii) optionally converting the vardenafil of Formula I to its pharmaceutically acceptable salts or hydrates.

Embodiments of the present invention include one or more of the following features. For example, the hydrogenation of the compound of Formula II to obtain the compound of Formula III or its salt is carried out using a transition metal catalyst.

The hydrogenation of the compound of Formula II to obtain the compound of Formula III or its salt is carried out in a solvent, which includes straight and branched chain alcohols, cyclic alcohols, aromatic alcohols, carboxylic acids or a mixture thereof.

Suitable straight and branched chain alcohols include methanol, ethanol, n-propanol or iso-propanol. Suitable cyclic alcohols include cyclopentanol or cyclohexanol. A suitable aromatic alcohol includes benzyl alcohol. Suitable carboxylic acids include formic acid or acetic acid. In another general aspect, the present invention provides for a process for the preparation of vardenafil of Formula I

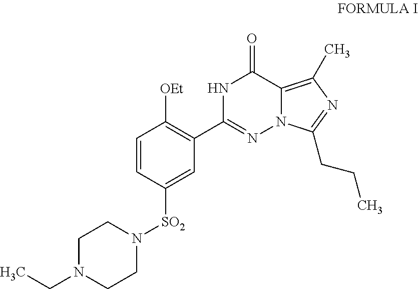

its pharmaceutically acceptable salts and hydrates, the process includes:
i) cyclizing N-{1-[3-{2-ethoxy-5-[(4-ethyl piperazin-1-yl)sulfonyl]phenyl}-5-oxo-4,5-dihydro-1,2,4-triazin-6-yl]ethyl}butanamide of Formula IV

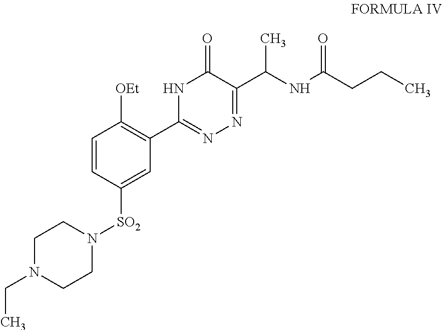

to obtain vardenafil of Formula I; and
ii) optionally converting the vardenafil of Formula I to its pharmaceutically acceptable salts or hydrates.

Embodiments of the present invention may include one or more of the following features. For example the cyclization of the compound of Formula IV to obtain the vardenafil of Formula I is carried out in the presence of a cyclizing agent comprising phosphorus oxychloride, oxalyl chloride or acetyl chloride.

The cyclization of the compound of Formula IV to obtain the vardenafil of Formula I is carried out in a solvent, which includes ethers, chlorinated hydrocarbons, ketones, esters, alcohols or a mixture thereof.

Suitable ethers include diethyl ether, diisopropyl ether, or tetrahydrofuran. Suitable chlorinated hydrocarbons include chloroform, dichloromethane, or 1,2-dichloroethane. Suitable ketones include acetone, methyl ethyl ketone or methyl isobutyl ketone. Suitable esters include methyl acetate, ethyl acetate, propyl acetate, or butyl acetate. Suitable alcohols include methanol, ethanol, n-propanol, or iso-propanol.

In another general aspect, the present invention provides for a process for the preparation of vardenafil of Formula I

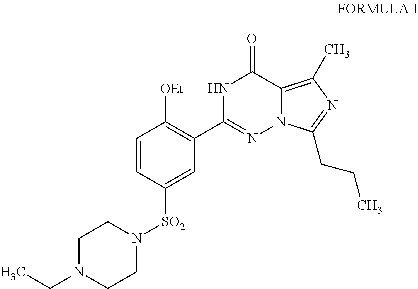

its pharmaceutically acceptable salts and hydrates, the process includes:
i) treating 2-ethoxy-5-(4-ethyl-1-piperazinyl sulfonyl) benzonitrile of Formula V

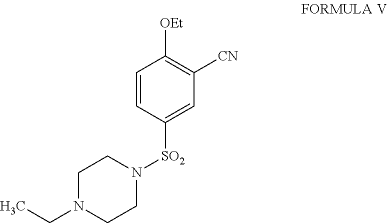

with hydroxylamine hydrochloride to obtain 2-ethoxy-N-hydroxy-5-[(4-ethylpiperazin-1-yl)sulphonyl]benzene carboximidamidine of Formula II;

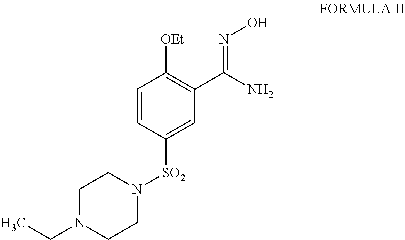

ii) hydrogenating the 2-ethoxy-N-hydroxy-5-[(4-ethylpiperazin-1-yl)sulphonyl]benzene carboximidamidine of Formula II to obtain 2-ethoxy-5-(4-ethyl-1-piperazinylsulfonyl)benzamidine of Formula III

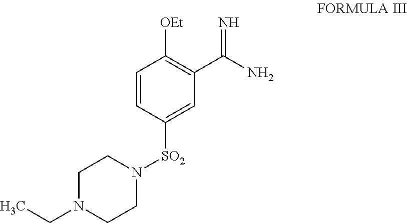

or its salt;
iii) treating the 2-ethoxy-5-(4-ethyl-1-piperazinylsulfonyl)benzamidine of Formula III or its salt with hydrazine hydrate to obtain 2-ethoxy-5-[(4-ethyl-1-piperazinylsulphonyl)benzene carboximido hydrazide of Formula VI;

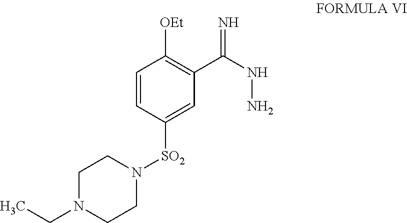

iv) reacting the 2-ethoxy-5-[(4-ethyl-1-piperazinylsulphonyl)benzene carboximido hydrazide of Formula VI with ethyl-3-(butanoylamino)-2-oxobutanoate of Formula VII;

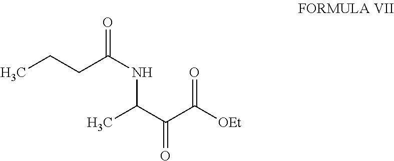

to obtain N-{1-[3-{2-ethoxy-5-[(4-ethyl piperazin-1-yl)sulfonyl]phenyl}-5-oxo-4,5-dihydro-1,2,4-triazin-6-yl]ethyl}butanamide of Formula IV

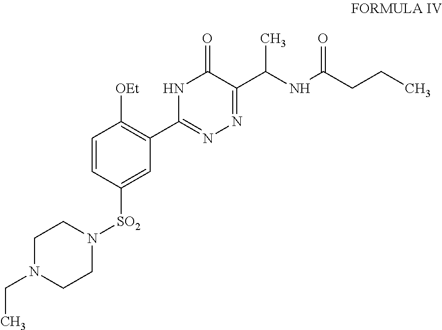

v) cyclizing the N-{1-[3-{2-ethoxy-5-[(4-ethyl piperazin-1-yl)sulfonyl]phenyl}-5-oxo-4,5-dihydro-1,2,4-triazin-6-yl]ethyl}butanamide of Formula IV to obtain the vardenafil of Formula I; and
vi) optionally converting the vardenafil of Formula I to its pharmaceutically acceptable salts or hydrates.

Embodiments of the invention may include one or more of the following features. For example, the reaction of the compound of Formula V with hydroxylamine hydrochloride to obtain the compound of Formula II is carried out in the presence of a base.

The base includes an organic base or an inorganic base. Suitable organic bases include triethylamine, diisopropylethylamine or 4-methyl morpholine. Suitable inorganic bases include potassium carbonate, sodium carbonate, sodium bicarbonate, lithium hydroxide monohydrate or lithium carbonate.

The reaction of the compound of Formula V with hydroxylamine hydrochloride to obtain the compound of Formula II is carried out in a solvent, which includes ethers, chlorinated hydrocarbons, ketones, esters, alcohols or a mixture thereof.

Suitable ethers include diethyl ether, diisopropyl ether, or tetrahydrofuran. Suitable chlorinated hydrocarbons include chloroform, dichloromethane, or 1,2-dichloroethane. Suitable ketones include acetone, methyl ethyl ketone or methyl isobutyl ketone. Suitable esters include methyl acetate, ethyl acetate, propyl acetate, or butyl acetate. Suitable alcohols include methanol, ethanol, n-propanol, or iso-propanol.

The hydrogenation of the compound of Formula II to obtain the compound of Formula III or its salt is carried out using a transition metal catalyst.

The hydrogenation of the compound of Formula II to obtain the compound of Formula III or its salt is carried out in a solvent which includes straight and branched chain alcohols, cyclic alcohols, aromatic alcohols, carboxylic acids or a mixture thereof.

Suitable straight and branched chain alcohols include methanol, ethanol, n-propanol or iso-propanol. Suitable cyclic alcohols include cyclopentanol or cyclohexanol. A suitable aromatic alcohol is benzyl alcohol. Suitable carboxylic acids include formic acid or acetic acid.

The treatment of the compound of Formula III or its salt with hydrazine hydrate to obtain the compound of Formula VI is carried out in a solvent which includes ethers, chlorinated hydrocarbons, ketones, esters, alcohols or a mixture thereof.

Suitable ethers include diethyl ether, diisopropyl ether, or tetrahydrofuran. Suitable chlorinated hydrocarbons include chloroform, dichloromethane, or 1,2-dichloroethane. Suitable ketones include acetone, methyl ethyl ketone or methyl isobutyl ketone. Suitable esters include methyl acetate, ethyl acetate, propyl acetate, or butyl acetate. Suitable alcohols include methanol, ethanol, n-propanol, or iso-propanol.

The reaction of the compound of Formula VI with the compound of Formula VII to obtain the compound of Formula IV is carried out in a solvent, which includes ethers, chlorinated hydrocarbons, ketones, esters, alcohols or a mixture thereof.

Suitable ethers include diethyl ether, diisopropyl ether, or tetrahydrofuran. Suitable chlorinated hydrocarbons include chloroform, dichloromethane, or 1,2-dichloroethane. Suitable ketones include acetone, methyl ethyl ketone or methyl isobutyl ketone. Suitable esters include methyl acetate, ethyl acetate, propyl acetate, or butyl acetate. Suitable alcohols include methanol, ethanol, n-propanol, or iso-propanol.

The cyclization of the compound of Formula IV to obtain the vardenafil of Formula I is carried out in the presence of a cyclizing agent which includes phosphorus oxychloride, oxalyl chloride or acetyl chloride.

The cyclization of the compound of Formula IV to obtain the vardenafil of Formula I is carried out in a solvent, which includes ethers, chlorinated hydrocarbons, ketones, esters, alcohols or a mixture thereof.

Suitable ethers include diethyl ether, diisopropyl ether, or tetrahydrofuran. Suitable chlorinated hydrocarbons include chloroform, dichloromethane, or 1,2-dichloroethane. Suitable ketones include acetone, methyl ethyl ketone or methyl isobutyl ketone. Suitable esters include methyl acetate, ethyl acetate, propyl acetate, or butyl acetate. Suitable alcohols include methanol, ethanol, n-propanol, or iso-propanol.

In another general aspect, the present invention provides for a compound selected from 2-ethoxy-5-(4-ethyl-1-piperazinylsulfonyl)benzamidine, 2-ethoxy-5-(4-ethyl-1-piperazinylsulfonyl)benzamidine tetraacetate, N-{1-[3-{2-ethoxy-5-[(4-ethyl piperazin-1-yl)sulfonyl]phenyl}-5-oxo-4,5-dihydro-1,2,4-triazin-6-yl]ethyl}butanamide or 2-ethoxy-5-[(4-ethyl-1-piperazinylsulphonyl)benzene carboximido hydrazide.

In an final general aspect, the present invention provides for the use of a compound selected from 2-ethoxy-5-(4-ethyl-1-piperazinylsulfonyl)benzamidine, 2-ethoxy-5-(4-ethyl-1-piperazinylsulfonyl)benzamidine tetraacetate, N-{1-[3-{2-ethoxy-5-[(4-ethyl piperazin-1-yl)sulfonyl]phenyl}-5-oxo-4,5-dihydro-1,2,4-triazin-6-yl]ethyl}butanamide or 2-ethoxy-5-[(4-ethyl-1-piperazinylsulphonyl)benzene carboximido hydrazide for the preparation of vardenafil, its pharmaceutically acceptable salts and hydrates

### DETAILED DESCRIPTION OF THE INVENTION

Pharmaceutically acceptable salts of vardenafil of Formula I may be formed by reaction with inorganic acids, organic acids, metals, ammonia or organic amines. Examples of inorganic acids include hydrochloric acid, hydrobromic acid, phosphoric acid, sulphuric acid, preferably hydrochloric acid. Examples of organic acids include carboxylic acids, and sulphonic acids. Examples of carboxylic acids include acetic acid, maleic acid, fumaric acid, malic acid, citric acid, tartaric acid, lactic acid, and benzoic acid. Examples of sulphonic acids include methanesulphonic acid, ethanesulphonic acid, phenylsulphonic acid, toluenesulphonic acid, and naphthalenedisulphonic acid. Examples of metals include sodium, potassium, magnesium, and calcium. Examples of organic amines include ethylamine, diethylamine, triethylamine, diethanolamine, triethanolamine, dicyclohexylamine, dimethylaminoethanol, arginine, lysine, ethylenediamine, and 2-phenylethylamine. The pharmaceutically acceptable salts of vardenafil of Formula I may be prepared by conventional means, such as, by treating with an appropriate acid or base or a salt thereof.

Hydrates of vardenafil of Formula I or its pharmaceutically acceptable salts may contain 1 to 5 equivalents of water in the crystal. The hydrates may be prepared by crystallizing from water or a solvent-water mixture. A preferred hydrate is vardenafil hydrochloride trihydrate.

The 2-ethoxy-5-(4-ethyl-1-piperazinyl sulfonyl)benzonitrile of Formula V, is used as an intermediate for the preparation of vardenafil of Formula I, pharmaceutically acceptable salts and hydrates thereof and may be prepared by the reactions known in the literature, such as those described in WO 01/98284.

In general, the 2-ethoxy-5-(4-ethyl-1-piperazinyl sulfonyl)benzonitrile of Formula V may be prepared by the sulphonylation of 2-ethoxy benzonitrile followed by the reaction of the 5-chlorosulphonyl-2-ethoxy benzonitrile with N-ethyl piperazine.

The sulphonylation of 2-ethoxy benzonitrile is carried out by reacting 2-ethoxy benzonitrile with chlorosulphonic acid. The reaction may be carried out below room temperature, preferably at about 0° C. to about 25° C., more preferably at about 5° C. to about 7° C.

The sulphonylation may also be carried out by reacting 2-ethoxy benzonitrile with sulphuric acid to obtain the sulphonic acid salt followed by reaction of the sulphonic acid salt with thionyl chloride.

The reaction of 5-chlorosulphonyl-2-ethoxy benzonitrile with N-ethyl piperazine may be carried out in a suitable solvent. Suitable solvents include ethers, chlorinated hydrocarbons, ketones, esters, alcohols or a mixture thereof. Examples of ethers include diethyl ether, diisopropyl ether, and tetrahydrofuran. Examples of chlorinated hydrocarbons include chloroform, and dichloromethane, 1,2-dichloroethane. Examples of ketones include acetone, methyl ethyl ketone, and methyl isobutyl ketone. Examples of esters include methyl acetate, ethyl acetate, propyl acetate, and butyl acetate. Examples of alcohol include methanol, ethanol, n-propanol, and iso-propanol. Preferably, a chlorinated hydrocarbon, such as, dichloromethane is used.

The reaction of 5-chlorosulphonyl-2-ethoxy benzonitrile with N-ethyl piperazine may be carried out by stirring at a temperature below room temperature. Preferably, stirring may be carried out at about 0° C. to about 25° C., with a preferred temperature of about 5° C. to about 10° C. Stirring may be carried out for about 1 hour to about 5 hours, preferably for about 2 hours.

The reaction of the 2-ethoxy-5-(4-ethyl-1-piperazinyl sulfonyl)benzonitrile of Formula V with hydroxylamine hydrochloride is carried out in the presence of a suitable base. The suitable base includes an organic base or an inorganic base. Examples of organic base include triethylamine, diisopropylethylamine or 4-methyl morpholine. Examples of inorganic base include potassium carbonate, sodium carbonate, sodium bicarbonate, lithium hydroxide monohydrate or lithium carbonate. Preferably, triethylamine is used.

The reaction of the 2-ethoxy-5-(4-ethyl-1-piperazinyl sulfonyl) benzonitrile of Formula V with hydroxylamine hydrochloride is carried out in a suitable solvent. The suitable solvent for this reaction includes the solvents described for the reaction of 5-chlorosulphonyl-2-ethoxy benzonitrile with N-ethyl piperazine. Preferably, the suitable solvent is methanol. The reaction mixture may be refluxed for about 1 hour to about 5 hours, preferably for about 2 hours.

The hydrogenation of the 2-ethoxy-N-hydroxy-5-[(4-ethyl piperazin-1-yl)sulfonyl]benzene carboximidamidine of Formula II is carried out using a transition metal catalyst in a suitable solvent. The transition metal catalyst may be a supported transition metal catalyst or a salt of a transition metal. The supported transition metal catalyst includes raney nickel, rhodium, ruthenium, platinum, or palladium supported on carbon. The salts of transition metals include salts of platinum, rhodium, and the like. Preferably, a supported transition metal catalyst such as palladium supported on carbon may be used.

The hydrogenation reaction is carried out in a suitable solvent. The suitable solvent includes straight and branched chain alcohols, cyclic alcohols, aromatic alcohols, carboxylic acids, or a mixture thereof. Examples of straight and branched chain alcohols include methanol, ethanol, n-propanol, or iso-propanol. Examples of cyclic alcohols include cyclopentanol or cyclohexanol. Examples of aromatic alcohols include benzyl alcohol. Examples of carboxylic acids include formic acid or acetic acid. Preferably, hydrogenation is carried out in carboxylic acids, such as acetic acid.

The hydrogenation reaction is carried out at a temperature of about 50° C. to about 70° C.; with a preferred temperature of about 60° C. The hydrogenation reaction may be carried out for a period of about 8 hours to about 16 hours; preferably for about 12 hours.

The suitable solvent may be recovered from the reaction mixture. Water may be added. The pH of the reaction mixture may be adjusted by adding an aqueous solution of a base, including sodium hydroxide or potassium hydroxide; preferably an aqueous sodium hydroxide solution is used.

Salts of 2-ethoxy-5-(4-ethyl-1-piperazinylsulfonyl)benzamidine of Formula III may be selected from salts of 2-ethoxy-5-(4-ethyl-1-piperazinylsulfonyl)benzamidine formed with organic and inorganic acids. Examples of organic acids include acetic acid, maleic acid, fumaric acid, malic acid, citric acid, tartaric acid, lactic acid or benzoic acid. Examples of inorganic acids include hydrochloric acid, hydrobromic acid, phosphoric acid or sulphuric acid.

The conversion of the 2-ethoxy-5-(4-ethyl-1-piperazinylsulfonyl)benzamidine of Formula III or its salts to the 2-ethoxy-5-[(4-ethyl-1-piperazinylsulphonyl)benzene rboximido hydrazide of Formula VI may be carried out by a reaction with hydrazine hydrate in a suitable solvent followed by dehydration. In a preferred embodiment, 2-ethoxy-5-(4-ethyl-1-piperazinylsulfonyl)benzamidine tetraacetate may be reacted with hydrazine hydrate in a suitable solvent followed by dehydration to obtain 2-ethoxy-5-[(4-ethyl-1-piperazinylsulphonyl)benzene carboximido hydrazide of Formula VI.

The suitable solvent may include those solvents described for the reaction of 5-chlorosulphonyl-2-ethoxy benzonitrile with N-ethyl piperazine. Preferably, an alcohol, such as ethanol, is used.

Dehydration may be carried out by refluxing in the presence of a dehydrating agent selected from magnesium sulphate, sodium sulphate, molecular sieves or by azeotropic distillation. Preferably, magnesium sulphate is used.

The ethyl-3-(butanoylamino)-2-oxobutanoate of Formula VII, used for the preparation of N-{1-[3-{2-ethoxy-5-[(4-ethyl piperazin-1-yl)sulfonyl]phenyl}-5-oxo-4,5-dihydro-1,2,4-triazin-6-yl]ethyl}butanamide of Formula IV, may be obtained by the processes reported in literature such as those described in Org. Process Res. Dev., 9(1), pages 88-97, (2005).

The 2-ethoxy-5-[(4-ethyl-1-piperazinylsulphonyl)benzene carboximido hydrazide of Formula VI may be isolated from the reaction mixture and used in the next step or the reaction mixture may be used as such in the next step without isolation.

The reaction of 2-ethoxy-5-[(4-ethyl-1-piperazinylsulphonyl)benzene carboximido hydrazide of Formula VI with ethyl-3-(butanoylamino)-2-oxobutanoate of Formula VII may be carried out in the presence of a suitable solvent.

The suitable solvent includes the group of solvents described for the reaction of 5-chlorosulphonyl-2-ethoxy benzonitrile with N-ethyl piperazine. Preferably, an alcohol, such as ethanol, is used.

The reaction mixture may be refluxed for about 30 minutes to about 8 hours, preferably about 3 hours to about 4 hours, and then cooled. The suitable solvent can be recovered. The residue can be further purified. Preferably, the residue is purified using silica gel chromatography.

The eluent to be used for purification using silica gel chromatography includes a mixture of alkyl acetate and alcohol. The alkyl acetate includes ethyl acetate, n-propyl acetate or ethyl methyl acetate. Examples of alcohol include methanol, ethanol, n-propanol or iso-propanol. Preferably, a mixture of ethyl acetate and methanol is used.

The N-{1-[3-{2-ethoxy-5-[(4-ethyl piperazin-1-yl) sulfonyl]phenyl}-5-oxo-4,5-dihydro-1,2,4-triazin-6-yl]ethyl}butanamide of Formula IV may be cyclized in a suitable solvent. The cyclization may be carried out using cyclizing agents including phosphorus oxychloride, oxalyl chloride, and acetyl chloride; preferably phosphorus oxychloride is used.

The suitable solvent including those from the group of solvents described for the reaction of 5-chlorosulphonyl-2-ethoxy benzonitrile with N-ethyl piperazine. Preferably, a chlorinated hydrocarbons, such as 1,2-dichloroethane, is used.

Vardenafil of Formula I, prepared by the process of the present invention, may be further purified. The purification may be carried out by crystallization or by chromatography. Preferably, purification is carried out by crystallization.

The process of the invention provides vardenafil of high purity. Isolation may be accomplished by concentration, precipitation, cooling, filtration or centrifugation, or a combination thereof followed by drying.

In the foregoing section embodiments are described by way of examples to illustrate the process of invention. However, this is not intended in any way to limit the scope of the present invention. Several variants of the examples would be evident to persons ordinarily skilled in the art which are within the scope of the present invention.

### EXAMPLES

### Example 1

### Preparation of 2-Ethoxy-N-Hydroxy-5-[(4-Ethyl Piperazin-1-yl)Sulfonyl]Benzene Carboximidamidine

### Step-a: Preparation of 5-Chlorosulfonyl-2-Ethoxy Benzonitrile

2-ethoxy benzonitrile (25 g, 170 mmol) was added to an ice cold solution of chlorosulfonic acid (183.7 g, 1.57 mol) over a period of one hour. The temperature of the reaction mixture was maintained at about 5° C. to 7° C. The reaction mixture was stirred at about 5° C. to 7° C. overnight. The reaction mass was added into ice water (500 mL) slowly at 10° C. to 15° C. The suspension was stirred for about 1 hour, filtered under nitrogen atmosphere to obtain 5-chlorosulfonyl-2-ethoxy benzonitrile as yellow solid which was used directly in next step.

### Step-b: Preparation of 2-Ethoxy-5-(4-Ethyl-1-Piperazinyl Sulfonyl)Benzonitrile

5-chlorosulfonyl-2-ethoxy benzonitrile obtained in step-a, was taken in dichloromethane (100 mL). The reaction mixture was cooled to about 0° C. to 5° C. N-ethyl piperazine (42.7 g, 37.3 mmol) was added dropwise over a period of 1 hour. The reaction mixture was stirred for about 2 hours at about 5° C. to 10° C. Dichloromethane was recovered under reduced pressure to obtain 2-ethoxy-5-(4-ethyl-1-piperazinyl sulfonyl) benzonitrile which was used directly in next step.

¹H NMR (CDCl₃): 1.03 (t, 3H), 1.53 (t, 3H), 2.43 (q, 2H), 2.53 (m, 4H), 3.03 (m, 4H), 4.24 (q, 2H), 7.07 (d, 1H), 7.88 (d, 1H), 7.94 (s, 1H)

M/Z=324 (M+H)⁺

### Step-c: Preparation of 2-Ethoxy-N-Hydroxy-5-[(4-Ethyl Piperazin-1-yl)Sulfonyl]Benzene Carboximidamidine

Hydroxylamine hydrochloride (59 g, 848 mmol) was added to the solution of 2-ethoxy-5-(4-ethyl-1-piperazinyl sulfonyl) benzonitrile in methanol (125 mL) at room temperature. Triethylamine (86.03 g, 850 mmol) was added slowly. The solution was refluxed for about 2 hours. Methanol was recovered under reduced pressure. Water (100 mL) was added. The reaction mixture was extracted with chloroform. The pH of aqueous layer was adjusted to between 8 to 9 by adding an aqueous sodium hydroxide solution. The reaction mixture was stirred for about 1 hour, filtered, washed with water and dried to obtain 2-ethoxy-N-hydroxy-5-[(4-ethyl piperazin-1-yl)sulfonyl]benzene carboximidamidine as a white solid (20.5 g).

Yield: 33% (for step a to c)

¹H NMR (CD₃OD): 1.05 (t, 3H), 1.45 (t, 3H), 2.46 (q, 2H), 2.57 (m, 4H), 3.03 (m, 4H), 4.22 (q, 2H), 7.25 (d, 1H), 7.77-7.86 (m, 2H)

M/Z=357(M+H)⁺

### Example 2

### Preparation of 2-Ethoxy-5-(4-Ethyl-1-Piperazinylsulfonyl)Benzamidine Tetraacetate

2-ethoxy-N-hydroxy-5-[(4-ethyl piperazin-1-yl)sulfonyl]benzene carboximidamidine (20 g, 56 mmol) was taken in acetic acid (100 mL) followed by the addition of 10% wet Palladium/Carbon (4 g). The reaction mixture was hydrogenated at about 60° C. for about 12 hours. After completion of the reaction, the catalyst was filtered through celite and washed with acetic acid (5 mL). Acetic acid was recovered under reduced pressure. Water (120 mL) was added to the reaction mass. The pH was adjusted to about 8 to 9 by adding an aqueous sodium hydroxide solution. The aqueous layer was washed with dichloromethane (100 mL). Water was recovered under reduced pressure. Diisopropyl ether (100 mL) was added. The contents were stirred for about 1 hour, filtered and dried under reduced pressure to obtain 2-ethoxy-5-(4-ethyl-1-piperazinylsulfonyl)benzamidine tetraacetate as white solid (15.6g).

Yield: 48.36%

¹H NMR (CD₃OD) 1.07 (t, 3H), 1.46 (t, 3H), 1.94 (s, 4*3H), 2.46 (q, 2H), 2.58 (m, 4H), 3.05 (m, 4H), 4.28 (q, 2H), 7.41 (d, 1H), 7.90 (s, 1H), 7.97 (d, 1H)

¹³C NMR (CD₃OD) 9.8, 13.34, 21.82, 44.91, 51.07, 51.41, 65.49, 113.27, 119.35, 127.19, 129.09, 133.66, 159.94, 164.42

M/Z=341(M+H)⁺

### Example 3

### Preparation of 2-Butyrylamino Propionic Acid

Butyryl chloride (71.7 g, 673.2 mmol) was added drop wise to a solution of D, L-alanine (50 g, 561.7 mmol) in aqueous sodium hydroxide (56 g, 1.4 mol) at about 5° C. to 10° C. The reaction mixture was stirred overnight at room temperature. The pH of the reaction mixture was adjusted to about 3 to 4 by adding concentrated hydrochloric acid. The reaction mixture was extracted with dichloromethane (3×300 mL). Dichloromethane was recovered to obtain an oily residue. The residue was crystallized from hexane (100 mL) to obtain 2-butyrylamino propionic acid as a white solid (34.5 g).

Yield: 38.7%

¹H NMR (CD₃OD): 0.94 (t, 3H), 1.38 (d, 3H), 1.59-1.67 (m, 2H), 2.2 (t, 2H), 4.38 (q, 1H)

M/Z: 160 (M+H)⁺

### Example 4

### Preparation of Ethyl-3-(Butanoylamino)-2-Oxobutanoate

Ethyl oxalyl chloride (85.84 g, 628 mmol) was added drop wise with stirring to a solution of 2-butyrylamino propionic acid (50 g, 314.4 mmol), pyridine (51.50 g, 660 mmol), and 4-dimethyl amino pyridine (1.25 g, 10 mmol) in tetrahydrofuran (200 mL). The reaction mixture was refluxed for about 3 to 4 hours, cooled, diluted with water (100 mL) and extracted with ethyl acetate (3×100 mL). Ethyl acetate was recovered to obtain an oily material. The oily material was dissolved in ethanol (100 mL). Sodium bicarbonate (15.8 g, 188 mmol) was added. The contents were refluxed for about 3 to 4 hours, cooled and sodium bicarbonate was removed by filtration. Ethanol was recovered under reduced pressure. The crude product was purified using silica gel chromatography eluting with hexane: ethyl acetate (3:1) to obtain ethyl-3-(butanoylamino)-2-oxobutanoate (21 g).

Yield: 31%

¹H NMR (CDCl₃): 0.95 (t, 3H), 1.36-1.43 (2t, 6H), 1.63-1.69 (m, 2H), 2.2 (t, 2H), 4.36 (q, 1H), 6.34 (bs, 1H)

M/Z: 216.2 (M+H)⁺

### Example 5

### Preparation of N-{1-[3-{2-Ethoxy-5-[(4-Ethyl Piperazin-1-yl) Sulfonyl]phenyl}-5-Oxo4,5-Dihydro-1,2,4-Triazin-6-yl]Ethyl}Butanamide

To a solution of 2-ethoxy-5-[(4-ethyl-1-piperazinyl sulfonyl) benzamidine tetraacetate (4 g, 6.8 mmol) in ethanol (15 mL) was added a solution of hydrazine hydrate (0.345 g, 6.8 mmoeel) in ethanol (5 mL) over about 10 to 15 minutes. The reaction mixture was stirred at room temperature for about 10 minutes. Magnesium sulfate (1 g) was added. The reaction mixture was heated to reflux. A solution of ethyl-3-(butanoylamino)-2-oxo butanoate (1.48 g, 6.8 mmol) in ethanol (10 mL) was added in about 15 to 20 minutes. The reaction mixture was stirred for about 3 to 4 hours at reflux temperature, cooled and filtered. Ethanol was recovered under reduced pressure. The residue was purified using silica gel chromatography eluting with ethyl acetate: methanol (9:1) to obtain N-{1-[3-{2-ethoxy-5-[(4-ethyl piperazin-1-yl)sulfonyl]phenyl}-5-oxo4,5-dihydro-1,2,4-triazin-6-yl]ethyl}butanamide (950 mg).

Yield: 27.14%

¹H NMR (CDCl₃): 0.94 (t, 3H), 1.02 (t, 3H), 1.54 (d, 2H), 1.65 (m, 2×3H), 2.19 (m, 2H), 2.40 (d, 2H), 2.52 (m, 4H), 3.07 (m, 4H), 4.42 (m, 2H), 5.27 (m, 1H), 6.85 (d, 1H), 7.18 (d, 1H), 7.85 (d, 1H), 8.85 (bs, 1H)

M/Z: 507.6 (M+H)⁺

### Example 6

### Preparation of 2-[2-Ethoxy-5-(4-Ethyl-Piperazine-1-Sulphonyl)-Phenyl]-5-Methyl-7-Propyl-3H-Imidazo[5-1-f][1,2,4]Triazin-4-one (Vardenafil)

To a solution of N-{1-[3-{2-ethoxy-5-[(4-ethyl piperazin-1-yl)sulfonyl]phenyl}-5-oxo4,5-dihydro-1,2,4-triazin-6-yl]ethyl}butanamide (0.7 g, 1.38 mmol) in 1,2-dichloroethane (10 mL) was added phosphorous oxychloride (1.67 g, 11 mmol). The reaction mixture was refluxed for about 2 hours. After completion of the reaction, the reaction mixture was cooled to room temperature, diluted with dichloromethane (20 mL) and neutralized by adding aqueous sodium hydroxide solution. The residue obtained by evaporating organic layer under reduced pressure was crystallized from diisopropylether (15 mL) to obtain vardenafil as white solid (0.3 g).

Yield: 45.8%®

¹H NMR (CDCl₃): 1.01(t, 3H), 1.06(t, 3H), 1.57 (t, 3H), 1.87 (q, 2H), 2.48 (m, 2H), 2.60 (m, 4H), 2.63 (s, 3H), 3.0 (t, 2H), 3.1 (m, 4H), 4.31 (q, 2H), 7.12 (d, 1H), 7.82 (dd, 1H), 8.4 (s, 1H), 9.75 (bs, 1H)

M/Z: 489.7 (M+H)⁺

1. A process for the preparation of vardenafil of Formula I, 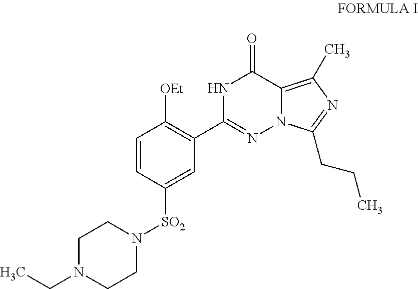
its pharmaceutically acceptable salts and hydrates, comprising:
i) hydrogenating 2-ethoxy-N-hydroxy-5-[(4-ethylpiperazin-1-yl)sulfonyl]benzene carboximidamidine of Formula II 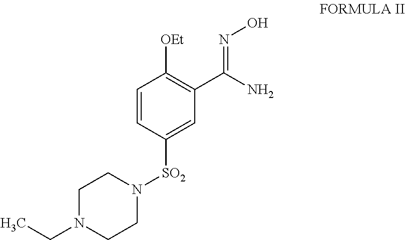
to obtain 2-ethoxy-5-[(4-ethyl-1-piperazinyl)sulfonyl]benzamidine of Formula III 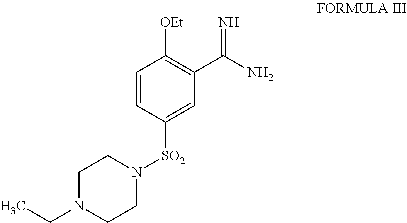
or its salt;
ii) converting the 2-ethoxy-5-[(4-ethyl-1-piperazinyl)sulfonyl]benzamidine of Formula III or its salt to vardenafil of Formula I; and
iii) optionally converting the vardenafil of Formula I to its pharmaceutically acceptable salts or hydrates.

2. The process of claim 1, wherein the hydrogenation of the compound of Formula II to obtain the compound of Formula III or its salt is carried out using a transition metal catalyst.

3. The process of claim 1, wherein the hydrogenation of the compound of Formula II to obtain the compound of Formula III or its salt is carried out in a solvent comprising straight and branched chain alcohols, cyclic alcohols, aromatic alcohols, carboxylic acids or a mixture thereof.

4. The process of claim 3, wherein the straight and branched chain alcohols comprise methanol, ethanol, n-propanol or iso-propanol.

5. The process of claim 3, wherein the cyclic alcohols comprise cyclopentanol or cyclohexanol.

6. The process of claim 3, wherein the aromatic alcohol comprises benzyl alcohol.

7. The process of claim 3, wherein the carboxylic acids comprise formic acid or acetic acid.

8. A process for the preparation of vardenafil of Formula I 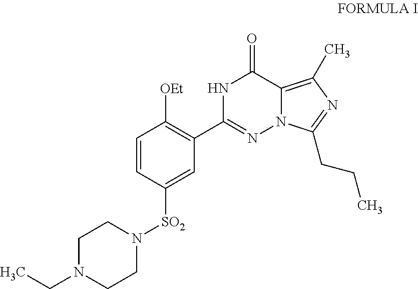
its pharmaceutically acceptable salts and hydrates, comprising:
i) treating 2-ethoxy-5-[(4-ethyl-1-piperazinyl)sulfonyl]benzonitrile of Formula V 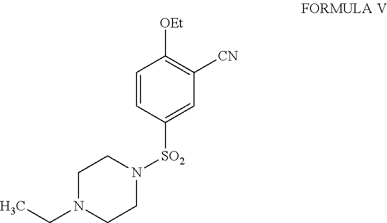
with hydroxylamine hydrochloride to obtain 2-ethoxy-N-hydroxy-5-[(4-ethylpiperazin-1-yl)sulfonyl]benzene carboximidamidine of Formula II; 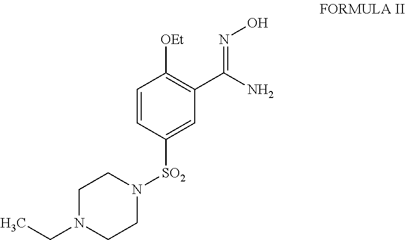
ii) hydrogenating the 2-ethoxy-N-hydroxy-5-[(4-ethylpiperazin-1-yl)sulfonyl]benzene carboximidamidine of Formula II to obtain 2-ethoxy-5-[(4-ethyl-1-piperazinyl)sulfonyl]benzamidine of Formula III 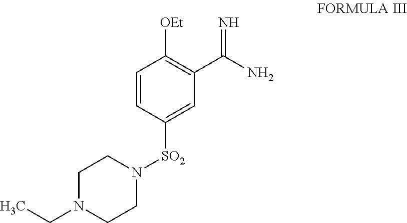
or its salt;
iii) treating the 2-ethoxy-5-[(4-ethyl-1-piperazinyl)sulfonyl]benzamidine of Formula III or its salt with hydrazine hydrate to obtain 2-ethoxy-5-[(4-ethyl-1-piperazinyl)sulfonyl]benzene carboximido hydrazide of Formula VI; 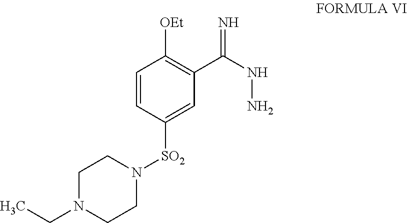
iv) reacting the 2-ethoxy-5-[(4-ethyl-1-piperazinyl)sulfonyl]benzene carboximido hydrazide of Formula VI with ethyl-3-(butanoylamino)-2-oxobutanoate of Formula VII; 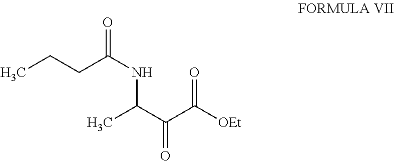
to obtain N-{1-[3-{2-ethoxy-5-[(4-ethylpiperazin-1-yl)sulfonyl]phenyl}-5-oxo-4,5-dihydro-1,2,4-triazin-6-yl]ethyl}butanamide of Formula IV 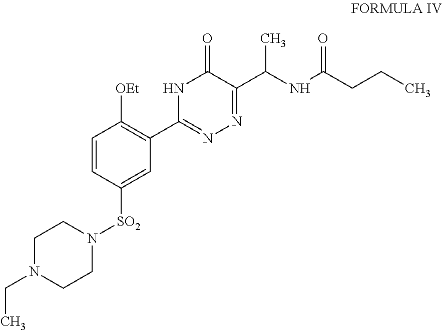
v) cyclizing the N-{1-[3-{2-ethoxy-5-[(4-ethylpiperazin-1-yl)sulfonyl]phenyl}-5-oxo-4,5-dihydro-1,2,4-triazin-6-yl]ethyl}butanamide of Formula IV to obtain the vardenafil of Formula I; and
vi) optionally converting the vardenafil of Formula I to its pharmaceutically acceptable salts or hydrates.

9. (canceled)

10. (canceled)

11. (canceled)

12. (canceled)

13. (canceled)

14. (canceled)

15. (canceled)

16. (canceled)

17. The process of claim 8, wherein the reaction of the compound of Formula V with hydroxylamine hydrochloride to obtain the compound of Formula II is carried out in the presence of a base.

18. The process of claim 9, wherein the base comprises an organic base or an inorganic base.

19. The process of claim 10, wherein the organic base comprises triethylamine, diisopropylethylamine or 4-methyl morpholine.

20. The process of claim 10, wherein the inorganic base comprises potassium carbonate, sodium carbonate, sodium bicarbonate, lithium hydroxide monohydrate or lithium carbonate.

21. The process of claim 8, wherein the reaction of the compound of Formula V with hydroxylamine hydrochloride to obtain the compound of Formula II is carried out in a solvent comprising ethers, chlorinated hydrocarbons, ketones, esters, alcohols or a mixture thereof

22. The process of claim 21, wherein the ethers comprise diethyl ether, diisopropyl ether, or tetrahydrofuran.

23. The process of claim 21, wherein the chlorinated hydrocarbons comprise chloroform, dichloromethane, or 1,2-dichloroethane.

24. The process of claim 21, wherein the ketones comprise acetone, methyl ethyl ketone or methyl isobutyl ketone.

25. The process of claim 21, wherein the esters comprise methyl acetate, ethyl acetate, propyl acetate, or butyl acetate.

26. The process of claim 21, wherein the alcohols comprise methanol, ethanol, n-propanol, or iso-propanol.

27. The process of claim 8, wherein the hydrogenation of the compound of Formula II to obtain the compound of Formula III or its salt is carried out using a transition metal catalyst.

28. The process of claim 8, wherein the hydrogenation of the compound of Formula II to obtain the compound of Formula III or its salt is carried out in a solvent comprising straight and branched chain alcohols, cyclic alcohols, aromatic alcohols, carboxylic acids or a mixture thereof.

29. The process of claim 28, wherein the straight and branched chain alcohols comprise methanol, ethanol, n-propanol or iso-propanol.

30. The process of claim 28, wherein the cyclic alcohols comprise cyclopentanol or cyclohexanol.

31. The process of claim 28, wherein the aromatic alcohol comprises benzyl alcohol.

32. The process of claim 28, wherein the carboxylic acids comprise formic acid or acetic acid.

33. The process of claim 8, wherein the treatment of the compound of Formula III or its salt with hydrazine hydrate to obtain the compound of Formula VI is carried out in a solvent comprising ethers, chlorinated hydrocarbons, ketones, esters, alcohols or a mixture thereof.

34. The process of claim 33, wherein the ethers comprise diethyl ether, diisopropyl ether, or tetrahydrofuran.

35. The process of claim 33, wherein the chlorinated hydrocarbons comprise chloroform, dichloromethane, or 1,2-dichloroethane.

36. The process of claim 33, wherein the ketones comprise acetone, methyl ethyl ketone or methyl isobutyl ketone.

37. The process of claim 33, wherein the esters comprise methyl acetate, ethyl acetate, propyl acetate, or butyl acetate.

38. The process of claim 33, wherein the alcohols comprise methanol, ethanol, n-propanol, or iso-propanol.

39. The process of claim 8, wherein the reaction of the compound of Formula VI with the compound of Formula VII to obtain the compound of Formula IV is carried out in a solvent comprising ethers, chlorinated hydrocarbons, ketones, esters, alcohols or a mixture thereof

40. The process of claim 39, wherein the ethers comprise diethyl ether, diisopropyl ether, or tetrahydrofuran.

41. The process of claim 39, wherein the chlorinated hydrocarbons comprise chloroform, dichloromethane, or 1,2-dichloroethane.

42. The process of claim 39, wherein the ketones comprise acetone, methyl ethyl ketone or methyl isobutyl ketone.

43. The process of claim 39, wherein the esters comprise methyl acetate, ethyl acetate, propyl acetate, or butyl acetate.

44. The process of claim 39, wherein the alcohols comprise methanol, ethanol, n-propanol, or iso-propanol.

45. (canceled)

46. (canceled)

47. (canceled)

48. (canceled)

49. (canceled)

50. (canceled)

51. (canceled)

52. (canceled)

53. (canceled)

54. The process of claim 8, wherein the cyclization of the compound of Formula IV to obtain the vardenafil of Formula I is carried out in the presence of a cyclizing agent comprising phosphorus oxychloride, oxalyl chloride or acetyl chloride.

55. The process of claim 8, wherein the cyclization of the compound of Formula IV to obtain the vardenafil of Formula I is carried out in a solvent comprising ethers, chlorinated hydrocarbons, ketones, esters, alcohols or a mixture thereof.

56. The process of claim 55, wherein the ethers comprise diethyl ether, diisopropyl ether, or tetrahydrofuran.

57. The process of claim 55, wherein the chlorinated hydrocarbons comprise chloroform, dichloromethane, or 1,2-dichloroethane.

58. The process of claim 55, wherein the ketones comprise acetone, methyl ethyl ketone or methyl isobutyl ketone.

59. The process of claim 55, wherein the esters comprise methyl acetate, ethyl acetate, propyl acetate, or butyl acetate.

60. The process of claim 55, wherein the alcohols comprise methanol, ethanol, n-propanol, or iso-propanol.
